(19) United States
(12) Patent Application Publication
(10) Pub. No.: US 2023/0267751 A1
(43) Pub. Date: Aug. 24, 2023

(54) VEHICLE SAFETY ASSISTANCE SYSTEM

(71) Applicants: Techno-Accel Networks Corp., Kobe-shi, Hyogo (JP); MURAKAMI CORPORATION, Shizuoka-shi, Shizuoka (JP)

(72) Inventors: Kazutami ARIMOTO, Kobe-shi, Hyogo (JP); Naoki YAMAUCHI, Kobe-shi, Hyogo (JP); Kagehisa KAJIWARA, Shizuoka-shi, Shizuoka (JP); Atsushi HAYAMI, Shizuoka-shi, Shizuoka (JP)

(73) Assignees: Techno-Accel Networks Corp., Kobe-shi, Hyogo (JP); MURAKAMI CORPORATION, Shizuoka-shi, Shizuoka (JP)

(21) Appl. No.: 18/017,701

(22) PCT Filed: Jul. 27, 2021
(86) PCT No.: PCT/JP2021/027812

§ 371 Date: Jan. 24, 2023

(30) Foreign Application Priority Data
Jul. 27, 2020 (JP) 2020-126791

(51) Int. Cl. G06V 20/59; B60Q 9/00; G08B 21/24; G06V 10/776; G06V 40/10; G06V 10/75; A61B 5/00; G01J 1/42

(52) U.S. Cl. CPC G06V 20/59; B60Q 9/00; G08B 21/24; G06V 10/776; G06V 40/10; G06V 10/75; A61B 5/6893; A61B 5/6892; G01J 1/42; B60R 25/31

(57) ABSTRACT

An object of the present invention is to provide a vehicle safety assistance system enabling perception of a state of passengers. A vehicle safety assistance system according to the present invention assists safety of at least one passenger on board a vehicle, and includes: a monitoring unit capable of monitoring the passenger in the vehicle; a recognition unit recognizing a state of the passenger by monitoring information from the monitoring unit; and a control unit carrying out control of operation of the vehicle or notification to the passenger on basis of recognition information from the recognition unit, in which the monitoring unit includes a monitoring camera.

### TECHNICAL FIELD

The present invention relates to a vehicle safety assistance system.

### BACKGROUND ART

A vehicle mounted with a sensor and a camera for perceiving an outboard situation in order to ensure safe travel of the vehicle has been known (for example, see Japanese Unexamined Patent Application Publication No. 2014-85331). The vehicle disclosed in the above publication is capable of recognizing a space on a road shoulder through use of an ultrasonic sensor, a radar, and a video camera, to safely pull over to the road shoulder and stop.

### PRIOR ART DOCUMENTS

### Patent Documents

Patent Document 1: Japanese Unexamined Patent Application Publication No. 2014-85331

### SUMMARY OF THE INVENTION

### Problems to be Solved by the Invention

On the other hand, fatal accidents involving young children and pets due to all passengers having gotten off vehicles, leaving the young children and pets inside, have been reported. In addition, deterioration of a health state and/or the like of a passenger, in particular a driver, during travel of a vehicle may lead to an accident. A vehicle safety assistance system against such accidents is also demanded.

The present invention was made in view of the foregoing circumstances, and an object of the present invention is to provide a vehicle safety assistance system enabling perception of a state of passengers.

### Means for Solving the Problems

A vehicle safety assistance system according to an aspect of the present invention assists safety of at least one passenger on board a vehicle, and includes: a monitoring unit capable of monitoring the at least one passenger in the vehicle; a recognition unit recognizing a state of the passenger by monitoring information from the monitoring unit; and a control unit carrying out control of operation of the vehicle or notification to the passenger on basis of recognition information from the recognition unit, in which the monitoring unit includes a monitoring camera.

### Effects of the Invention

The vehicle safety assistance system according to the present invention enables perception of a state of a passenger.

### BRIEF DESCRIPTION OF THE DRAWINGS

FIG. 1 is a schematic view illustrating a configuration of a vehicle safety assistance system according to an embodiment of the present invention.

FIG. 2 is a schematic view illustrating a vehicle mounted with the vehicle safety assistance system of FIG. 1.

FIG. 3 is a schematic view illustrating a configuration of a monitoring unit of FIG. 1.

FIG. 4 is a schematic view illustrating a configuration of a recognition unit of FIG. 1.

FIG. 5 is a flow diagram showing a control method of the vehicle safety assistance system of FIG. 1.

### DESCRIPTION OF EMBODIMENTS

First, embodiments of the present invention are listed and described.

A vehicle safety assistance system according to an aspect of the present invention assists safety of at least one passenger on board a vehicle, and includes: a monitoring unit capable of monitoring the at least one passenger in the vehicle; a recognition unit recognizing a state of the at least one passenger by monitoring information from the monitoring unit; and a control unit carrying out control of operation of the vehicle or notification to the at least one passenger on basis of recognition information from the recognition unit, in which the monitoring unit includes a monitoring camera.

The vehicle safety assistance system monitors a passenger in the vehicle by the monitoring camera, recognizes his/her behavior by the recognition unit, and carries out control of operation of the vehicle or notification to the passenger by the control unit, whereby safe travel of the vehicle can be ensured.

It is preferred that: the monitoring unit includes a belongings identification means identifying belongings likely to be brought into the vehicle; the monitoring information includes belongings information identified by the belongings identification means; the recognition information includes belongings detection information identifying whether the belongings are present in the vehicle and getting-off information identifying whether all of the at least one passenger has gotten off the vehicle; and when it is identified that all of the at least one passenger has gotten off the vehicle on basis of the getting-off information and that the belongings are present in the vehicle on basis of the belongings detection information, the control unit notifies the at least one passenger that the belongings are present in the vehicle. In a case in which belongings brought into the vehicle and identified by the belongings identification means are still present in the vehicle even after all the passengers have gotten off the vehicle, the belongings can be identified as an object left behind in the vehicle. The object left behind can be inhibited by the control unit notifying the passenger of the belongings being present in the vehicle.

It is preferred that the monitoring unit includes a monitoring radar. Employing the monitoring radar in addition to the monitoring camera enables identification of, for example, a target object wrapped in a blanket or the like, whereby monitoring accuracy can be improved.

It is preferred that the belongings identification means uses periodic oscillation detected by the monitoring radar. In a case in which the target object is a living object, heartbeats and breathing thereof are detected by the monitoring radar as the periodic oscillation. Therefore, using the periodic oscillation detected by the monitoring radar enables identification of whether the target object is a living object or a non-living object. In addition, in a case in which cycles thereof, being the heartbeats and breathing, are within predetermined ranges, the target object can be inferred to be a young child. Therefore, using the periodic oscillation detected by the monitoring radar for identification of the belongings enables detection with high accuracy of a young child left behind.

It is preferred that the recognition unit includes: a quantification means expressing the state of the at least one passenger by a numerical value from the monitoring information through an evaluation function; an optimization means optimizing a threshold value for identifying the state of the at least one passenger according to a magnitude relationship with the numerical value; and a recognition information identification means identifying the state of the at least one passenger as the recognition information from the numerical value and the threshold value. By thus optimizing the threshold value for identifying the state of the passenger, identification accuracy of the state of the passenger can be improved. Note that optimization of the threshold value includes, in addition to a method of adjusting a numerical value of the threshold value itself, a method of adding a bias value to the evaluation function to relatively adjust the threshold value.

It is preferred that the monitoring unit includes a database on which a reference image of a passenger captured beforehand is recorded, and a passenger identification means identifying the passenger by comparatively searching for a captured image of the passenger captured by the monitoring camera with the reference image, in which the passenger identification means is configured to be capable of calculating a degree of matching between the captured image and the reference image; and the optimization means of the recognition unit uses the degree of matching. The degree of matching is considered to represent validity of individual distinguishment of the passenger, in other words, reliability of a captured video. By optimizing the threshold value by the optimization means through use of the degree of matching, identification accuracy of the state of the passenger can further be improved.

It is preferred that the monitoring unit includes a photometer measuring luminosity of a periphery of the vehicle; and the optimization means of the recognition unit uses the luminosity. The monitoring camera is considered to have lower distinguishing performance with lower luminosity, and the monitoring radar has relatively high identification performance with low luminosity. In other words, by the optimization means of the recognition unit adjusting the priority of information from the monitoring camera or the monitoring radar according to luminosity, identification accuracy of the state of the passenger can further be improved.

It is preferred that: the monitoring unit includes a vital sensor acquiring biological information of the at least one passenger; and the monitoring information includes the biological information obtained from the vital sensor. By thus including the biological information in the monitoring information, an accident involving the vehicle due to deterioration of the health state of the passenger can be inhibited.

It is preferred that: the monitoring unit includes an ultrasonic sensor capable of detecting a person approaching the vehicle from outside of the vehicle, and an outboard camera or an outboard radar for sensing the person, in which the monitoring information includes outboard information obtained from the ultrasonic sensor, and the outboard camera or the outboard radar; the recognition information includes suspicious person identification information identifying whether the person is a suspicious person from the outboard information; and It is preferred that the control unit uses the suspicious person identification information. By thus using the suspicious person identification information, prevention of damage of the vehicle or objects in the vehicle is enabled.

### DETAILS OF EMBODIMENTS OF INVENTION

Hereinafter, a vehicle safety assistance system according to an embodiment of the present invention is described with appropriate reference to the drawings.

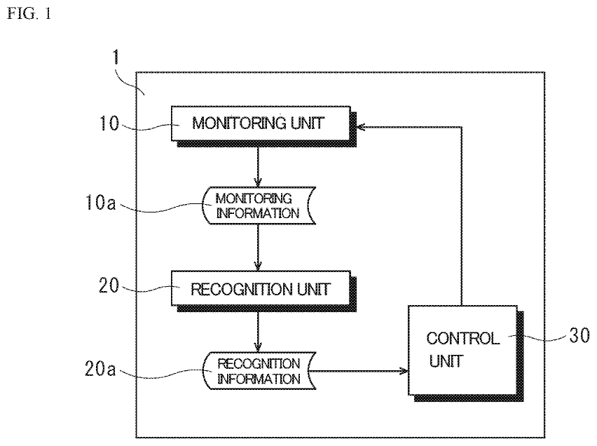

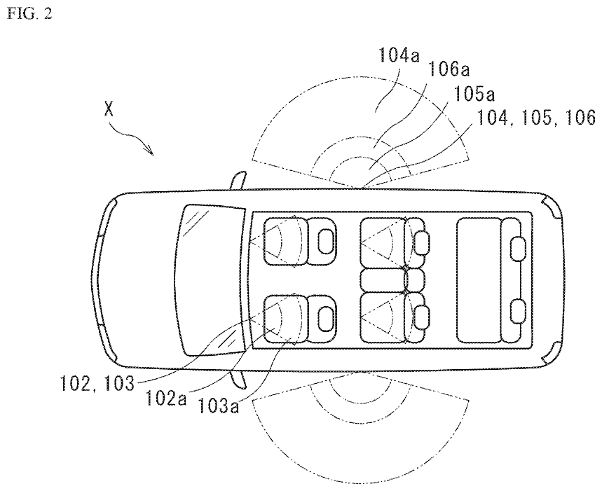

A vehicle safety assistance system 1 illustrated in FIG. 1 assists safety of at least one passenger on board a vehicle X illustrated in FIG. 2. The vehicle safety assistance system 1 includes: a monitoring unit 10 capable of monitoring the passenger in the vehicle X; a recognition unit 20 recognizing a state of the passenger by monitoring information 10a from the monitoring unit 10; and a control unit 30 carrying out control of operation of the vehicle X or notification to the passenger on the basis of recognition information 20a from the recognition unit 20.

### <Monitoring Unit>

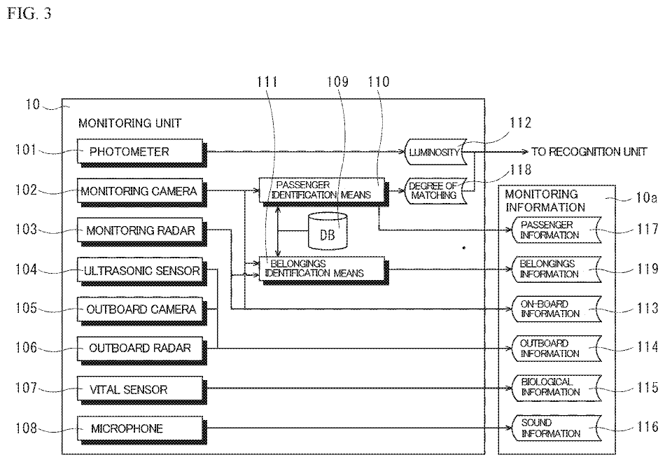

The monitoring unit 10 includes, as illustrated in FIG. 3, a photometer 101, a monitoring camera 102, a monitoring radar 103, an ultrasonic sensor 104, an outboard camera 105, an outboard radar 106, a vital sensor 107, a microphone 108, a database 109, a passenger identification means 110, and a belongings identification means 111.

### (Photometer)

The photometer 101 measures luminosity 112 of a periphery of the vehicle X. The photometer 101 may measure luminosity of the outside of the vehicle X, but preferably measures luminosity of the inside of the vehicle X. Since the vehicle safety assistance system 1 is configured to monitor principally the passenger in the vehicle X, using luminosity of the inside of the vehicle X enables improvement of the monitoring accuracy better. Note that the measured luminosity 112 is transmitted to the recognition unit 20.

### (Monitoring Camera and Monitoring Radar)

The monitoring camera 102 and the monitoring radar 103 are used for monitoring the passenger in the vehicle X, and for identification of the passenger and the belongings described later. The monitoring camera 102 identifies a target object by means of an image, while the monitoring radar 103 identifies a target object by means of reflected waves. The monitoring camera 102 is good at capturing a shape and movement of the target object, but cannot monitor when the target object is not visually recognizable. Therefore, employing the monitoring radar 103 in addition to the monitoring camera 102 enables identification of, for example, a target object wrapped in a blanket or the like, whereby monitoring accuracy can be improved.

The monitoring camera 102 and the monitoring radar 103 may monitor the entire living space of the vehicle X; however, it is preferred that a pair of the monitoring camera 102 and the monitoring radar 103 is provided for each passenger's seat as illustrated in FIG. 2. Providing a pair of the monitoring camera 102 and the monitoring radar 103 for each passenger enables improvement of the monitoring accuracy.

In a case in which the monitoring camera 102 and the monitoring radar 103 are provided for each passenger, a monitoring region 102a of the monitoring camera 102 is preferably a region centered on a position of a face of the passenger as illustrated in FIG. 2. With the monitoring camera 102, facial complexion, a facial angle, a sight line angle, the number of blinks, occurrence or non-occurrence of yawns, and the like of the passenger can be observed. On the other hand, a monitoring region 103a of the monitoring radar 103 is preferably a broad range including a periphery of the seat in light of improving detection accuracy of an object left behind, described later.

The monitoring information 10a includes on-board information 113 obtained by the monitoring camera 102 and the monitoring radar 103. By thus including the on-board information 113 in the monitoring information 10a, a state of the passenger can be comprehended.

### (Ultrasonic Sensor, Outboard Camera, and Outboard Radar)

The ultrasonic sensor 104, the outboard camera 105, and the outboard radar 106 monitor a person approaching the vehicle X. Specifically, the ultrasonic sensor 104 is capable of detecting a person approaching the vehicle X from outside. In addition, the outboard camera 105 and the outboard radar 106 are provided for sensing the person. Note that it is preferred that both the outboard camera 105 and the outboard radar 106 are employed in light of improving the sensing accuracy; however, either one of these enables the sensing. Therefore, either one of the outboard camera 105 and the outboard radar 106 may be omitted.

The ultrasonic sensor 104, the outboard camera 105, and the outboard radar 106 that monitor the person approaching the vehicle X may be arranged on each of both lateral faces of the vehicle X as illustrated in FIG. 2.

Since the ultrasonic sensor 104 is good at sensing an object at a relatively long distance, the monitoring region 104a of the ultrasonic sensor 104 is defined to be relatively broad, for example to sense a person at a distance of no less than 5 m and no greater than 20 m, and preferably no less than 10 m and no greater than 20 m. In addition, since the monitoring camera 102 senses the person by means of an image thereof, the monitoring region 105a is defined to be at a short distance enabling relatively clear sensing of the person, for example no greater than 5 m. The outboard radar 106 principally covers a distance in-between, and a monitoring region 106a thereof is defined to be at a distance of no less than 5 m and no greater than 10 m.

The monitoring information 10a includes outboard information 114 obtained from the ultrasonic sensor 104, the outboard camera 105, and the outboard radar 106. By thus including the outboard information 114 in the monitoring information 10a, whether the person is a suspicious person can be identified.

### (Vital Sensor)

The vital sensor 107 obtains biological information 115 of a passenger. Specifically, the vital sensor 107 can sense a pulse rate, a heart rate, a heartbeat interval, blood pressure, a blood glucose level, a breathing rate, and the like of the passenger. Among these, it is preferred to use the pulse rate and the breathing rate.

As the vital sensor 107, non-contact type vital sensors are preferred. Among these, a vital sensor employing a doppler sensor is particularly preferred in light of accuracy. In a case of using the non-contact type vital sensor 107, the vital sensor 107 can be installed in the same position as the monitoring camera 102 and the like. In this case, in light of ease of attachment to the vehicle X, the vital sensor 107 is preferably unitized with the monitoring camera 102 and the monitoring radar 103.

Alternatively, a contact type vital sensor may also be used as the vital sensor 107. As such a contact type vital sensor 107, a mat sensor has been known and may be attached to, for example, a surface of the seat of the vehicle X.

The monitoring information 10a includes the biological information 115 obtained from the vital sensor 107. By thus including the biological information 115 in the monitoring information 10a, accident of the vehicle X due to deterioration of the health state of the passenger can be inhibited.

### (Microphone)

The microphone 108 obtains words uttered by the passenger in the vehicle X as sound information 116.

The microphone 108 can be installed in the same position as the monitoring camera 102 and the like. In this case, in light of ease of attachment to the vehicle X, the microphone 108 is preferably be unitized with the monitoring camera 102 and the monitoring radar 103.

The monitoring information 10a includes the sound information 116 obtained from the microphone 108. By thus including the sound information 116 in the monitoring information 10a, an accident involving the vehicle X due to deterioration of the health state of the passenger can be inhibited.

### (Database)

On the database 109, a reference image of a passenger captured beforehand is recorded. In addition, a reference image of belongings likely to be brought into the vehicle X is recorded on the database 109.

The database 109 is configured with, for example, a well-known storage device, and data (reference images) thereof is referred to by a passenger identification means 110 and a belongings identification means 111 described later.

### (Passenger Identification Means)

The passenger identification means 110 identifies the passenger by comparatively searching for a captured image of the passenger captured by the monitoring camera 102 with the reference image of the passenger recorded in the database 109. In other words, passenger information 117 identifying who is seated in which seat can be obtained by the passenger identification means 110.

The passenger identification means 110 is embodied by, for example, a microcontroller. Alternatively, the passenger identification means 110 may be embodied by a dedicated matching circuit.

As a method for comparatively searching, either of: a method of providing a predetermined evaluation function and determining through magnitude of the evaluation function; or an inference model trained by machine learning, as generally referred to AI (artificial intelligence), can be employed. Note that, for inference using such an inference model, a well-known inference technique related to AI can be employed.

The monitoring information 10a includes the passenger information 117 obtained from the passenger identification means 110. Normal states of the passengers are individually different. By thus including the passenger information 117 in the monitoring information 10a, a health state of the passenger can be comprehended in consideration of the individual difference, whereby the monitoring accuracy can be improved.

In addition, the passenger identification means 110 is configured to be capable of calculating a degree of matching 118 between the captured image and the reference image. In a case in which an evaluation function is used for comparative search, the evaluation function being defined such that, for example, a value of the evaluation function is zero when the captured image and the reference image perfectly match and the value of the evaluation function increases as a difference increases, the value of the evaluation function itself can be used as the degree of matching 118. Note that the calculated degree of matching 118 is transmitted to the recognition unit 20.

### (Belongings Identification Means)

The belongings identification means 111 identifies belongings likely to be brought into the vehicle X.

The belongings identification means 111 is embodied by, for example, a microcontroller. The microcontroller embodying the passenger identification means 110 may also serve as this microcontroller. Alternatively, as in the case of the passenger identification means 110, the belongings identification means 111 may be embodied by a dedicated matching circuit.

The belongings are exemplified by a young child, a pet, a bag, a mobile phone, and the like. These are targets of detection, by the recognition unit 20 described above, of belongings still present in the vehicle even after all the passengers have gotten off the vehicle, i.e., an object left behind.

As a method for identifying the belongings, for example, a method of using a reference image of the belongings recorded in the database 109 can be adopted.

In this case, the fact that the belongings are brought into the vehicle may be detected beforehand through image analysis of the monitoring camera 102 (or the outboard camera 105) upon boarding of the passenger. Specifically, this can be embodied by a method using an evaluation function or a method using AI, as in the case of the passenger identification means 110. By thus detecting beforehand the fact that the belongings have been brought in, detection accuracy of the object left behind can be improved. In addition, it is preferred to use the monitoring radar 103 (or the outboard radar 106) in combination as described later, whereby the belongings not directly visually recognizable from outside and not easily recognized only with images may be detected.

Alternatively, an object carried by a passenger before boarding the vehicle X may be identified through image analysis of the outboard camera 105 or the monitoring camera 102, or by AI, and the identified object may be considered as the belongings. In this case, the need for the database 109 can be eliminated. Instead of the database 109, generation of metadata, being tag information for search, through extraction of, for example, characteristic features of the belongings (e.g., characteristic features such as red, rectangular, and the like in a case of a bag) is effective. In addition, metadata to which boarding information (place, time, and the like of boarding the vehicle X) is linked may be generated.

On the other hand, the belongings likely to be brought into the vehicle X upon boarding of the passenger may be read from the database 109. In this case, in regard to the belongings recorded in the database 109, exhaustive search is conducted as to whether the belongings are still present in the vehicle even after all the passengers have gotten off the vehicle. By thus conducting exhaustive search, detection accuracy of the object left behind can be improved.

In addition, the belongings may be changed according to the passenger to be on board the vehicle X, in other words, for each passenger identified by the passenger identification means 110.

The monitoring information 10a includes the belongings information 119 obtained from the belongings identification means 111. By thus including the belongings information 119 in the monitoring information 10a, detection of an object left behind is enabled.

It is preferred that the belongings identification means 111 uses periodic oscillation detected by the monitoring radar 103. In a case in which the target object is a living object, heartbeats and breathing thereof are detected by the monitoring radar 103 as the periodic oscillation. Therefore, using the periodic oscillation detected by the monitoring radar 103 enables identification of whether the target object is a living object or a non-living object. In addition, in a case in which a cycle thereof, being the heartbeats and breathing, is within a predetermined range, the target object can be inferred to be a young child. Therefore, using the periodic oscillation detected by the monitoring radar 103 for identification of the belongings enables detection with high accuracy of a young child left behind.

In a case of detecting a young child being left behind, it is preferred to detect boarding or nonboarding of a young child during boarding of the passenger. Since completion of boarding of the passenger can be determined by start of travel of the vehicle X, specifically it is preferred that the belongings identification means 111 uses periodic oscillation detected by the monitoring radar 103 after the start of travel of the vehicle X. In addition, for identification of a young child, it is preferred to use information from the vital sensor 107 in light of identification accuracy. By thus identifying the belongings by means of the periodic oscillation and the like after start of travel of the vehicle X, detection of a young child being left behind is enabled with higher accuracy.

### <Recognition Unit>

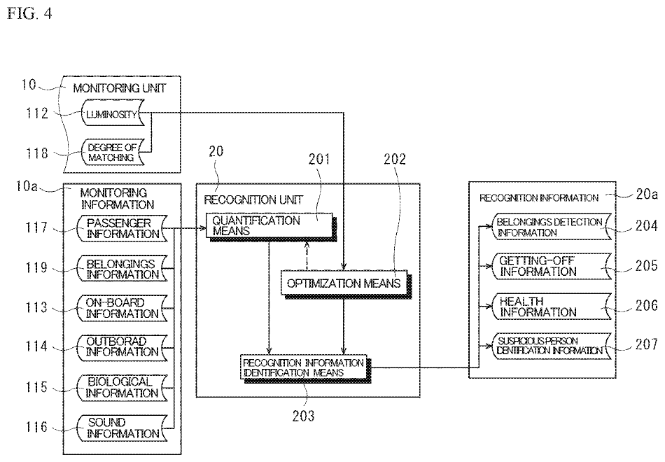

The recognition unit 20 includes, as illustrated in FIG. 4, a quantification means 201 expressing the state of the passenger by a numerical value from the monitoring information 10a through an evaluation function; an optimization means 202 optimizing a threshold value for identifying the state of the passenger according to a magnitude relationship with the numerical value; and a recognition information identification means 203 identifying the state of the passenger as the recognition information 20a from the numerical value and the threshold value.

By thus optimizing the threshold value for identifying the state of the passenger, identification accuracy of the state of the passenger can be improved. Hereinafter, the recognition information 20a recognized by the recognition unit 20 of the vehicle safety assistance system 1 is specifically described.

### (Detection of Object Left Behind)

The vehicle safety assistance system 1 can detect the belongings, which are identified by the belongings identification means 111 to be likely to be brought into the vehicle X, as an object left behind. In other words, the recognition information 20a includes belongings detection information 204 identifying whether the belongings are present in the vehicle X, and getting-off information 205 identifying whether all the passengers have gotten off the vehicle X. This is because, when it is identified that all the passengers have gotten off the vehicle X on the basis of the getting-off information 205 and that the belongings are present in the vehicle X on the basis of the belongings detection information 204, the belongings can be identified as an object left behind.

The belongings detection information 204 can be extracted by using the on-board information 113 of the monitoring information 10a, in other words information from the monitoring camera 102 and the monitoring radar 103.

An extraction method for extracting the belongings detection information 204 includes, for example, a coordinate axis addition step, a threshold value definition step, a synthesis step, and a determination step. The extraction method may be carried out by software, but is preferably carried out by hardware in light of processing speed.

In the coordinate axis addition step, camera data and radar data with an x-y coordinate being added are created respectively. By adding the x-y coordinate, overlapping of the camera data and the radar data is facilitated.

This processing is executed by the quantification means 201. Prior to addition of the x-y coordinate, preprocessing such as noise removal may be performed on the information from the monitoring camera 102 and the monitoring radar 103.

In the threshold value definition step, an adaptive bias value used in the synthesis step described later and/or a threshold value for the evaluation function used in the determination step described later is/are decided. This processing is executed by the optimization means 202.

The adaptive bias value as referred to means a weighting value for synthesizing a plurality of types of signals. In the belongings identification means 111, an evaluation function V is obtained by synthesizing camera data R and radar data C, in other words through data fusion. At this time, for example, the evaluation function V is calculated as in the following formula 1 with weighting variables a1 and a2. In this case, f(R) is an evaluation function (scalar) obtained from the camera data R, g(C) is an evaluation function (scalar) obtained from the radar data C, and a1 and a2 are adaptive bias values. By thus including a plurality of pieces of information (information from the monitoring camera 102 and the monitoring radar 103) in the monitoring information 10a and fusing these to provide an evaluation function, recognition accuracy of the state of the passenger can be improved.

V=a1×f(R)+a2×g(C) 1

It is preferred to use the luminosity 112 of the monitoring unit 10 for determination of the adaptive bias value. The monitoring camera 102 is considered to have lower distinguishing performance when the luminosity 112 is low, and the monitoring radar 103 has relatively high identification performance when the luminosity 112 is low. In other words, by adjusting priority of information from the monitoring camera 102 or the monitoring radar 103 according to the luminosity 112, and defining the weighting variables a1 and a2 such that, for example, a2 becomes greater when the luminosity 112 is lower, identification accuracy can be further improved as to whether the passenger has left an object behind.

It is preferred to use the degree of matching 118 of the monitoring unit 10 for determination of the adaptive bias value. The degree of matching 118 is considered to represent validity of individual distinguishment of the passenger, in other words, reliability of captured video. For example, when the degree of matching 118 is low, the distinguishing performance of the monitoring camera 102 can be considered to be lowered. For example, by optimizing the weighting variables a1 and a2 such that, for example, a2 becomes greater when the degree of matching 118 is lower, identification accuracy can be further improved as to whether the passenger has left an object behind. Note that the weighting variables a1 and a2 can be used in combination with the luminosity 112 and the degree of matching 118. Hereinafter, the same applies to other elements.

Alternatively, the weighting variable may be selected according to characteristic features of an image to be recognized. For example, when an image from the monitoring camera 102 included in the on-board information 113 is resized into an input format, e.g., resolution, suited to the optimization means 202, it is possible to determine whether the input image is a color image or a binary image (such as an infrared image). In this regard, it is preferred that a weighting variable for a color image and a weighting variable for a binary image are prepared beforehand, and are switched according to a result of the determination. This enables selection of an appropriate weighting variable according to the input image. The weighting variable may also be selected according to luminance, chroma saturation, or a combination thereof. In this case, three or more weighting variables may be prepared.

Alternatively, a value of the weighting variable may be changed according to a type of the belongings to be detected as an object left behind. For example, in a case of detecting a bag, weighting is preferably done such that information on a color matching the color of the bag observable by the monitoring camera 102 is more weighted. In addition, a shape and a size of the belongings to be detected may also be used. Note that, in these examples, optimization is not necessarily possible only with the weighting variables a1 and a2 in the above formula 1. For example, weighting of information on the color is made possible by using the following formula 2 with new weighting variables a1_{R}, a1_{G}, a1_{B} being added to the above formula 1 to divide RGB color information.

f(R)=a1_{R}×f_{R}(R)+a1_{G}×f_{G}(R)+a1_{B}×f_{B}(R) 2

The vehicle safety assistance system 1 is capable of detecting a young child left behind, by using the periodic oscillation detected by the monitoring radar 103 in the belongings identification means 111. Hereinafter, a method for detecting a young child is described.

Since the vehicle safety assistance system 1 includes the monitoring camera 102, it is easy to detect a young child as an object left behind when image recognition of the target young child is enabled by the monitoring camera 102. On the other hand, there is a case in which a young child cannot be detected by the monitoring camera 102, such as a case in which the young child is sleeping wrapped in a blanket or the like.

The vehicle safety assistance system 1 uses the monitoring radar 103 in such a case. When the monitoring radar 103 observes the young child sleeping wrapped in a blanket as described above, periodic oscillation is detected. The periodic oscillation is based on heartbeat and breathing of the young child. Therefore, the periodic oscillation is obtained by superposing periodic oscillations of the heartbeat and breathing of the young child. In addition, a heart rate and a breathing rate of young children are known to be within certain ranges. These ranges are different from those of typical adults and pets.

From the foregoing, it is possible to infer that a young child is present in a case in which the monitoring radar 103 detects periodic oscillation and the oscillation is determined to be constituted of two periods corresponding to the above-described heart rate and the breathing rate as a result of calculating the periods of the oscillation. A young child being left behind can thus be detected.

The evaluation function V of the above formula 1 obtained by synthesizing the camera data R and the radar data C can also be used for this detection of a young child being left behind. In regard to a1 and a2, which are adaptive bias values, for example it is preferred to use the evaluation function of the monitoring camera 102 with a2=0 when the detection can be made with the monitoring camera 102, and to control to increase the value of a2 when the detection cannot be made with the monitoring camera 102. Determination of whether the detection can be made with the monitoring camera 102 may be based on either a result in the monitoring camera 102, or environmental information. In this case, the environment information includes the luminosity 112 and blind spots of the monitoring camera 102. In addition, the environment information may include the biological information 115 obtained from the vital sensor 107.

Note that, for example, one of the heart rate and the breathing rate of the young child can be sensed by the vital sensor 107.

In the above-described synthesis step, image data is recognized through overlapping the camera data and the radar data, and a value of the evaluation function for identifying whether the belongings are present in the vehicle X is calculated. This processing is executed by the quantification means 201.

Specifically, the following procedure takes place. Image data is obtained through overlapping processing of the camera data and the radar data obtained in time series and assembled at a predetermined time interval. By thus carrying out the overlapping processing, for example a moving object (living object) and a non-moving object (non-living object) can be distinguished from each other.

Next, the image data having been subjected to the overlapping processing is subjected to compression processing, and a time-series correlation is extracted. By obtaining the time-series correlation, it can be detected that, for example, the belongings have moved to a blind spot of the monitoring camera 102 and the like, whereby optimization of the weighting variable is facilitated in the subsequent matching processing.

Then, the matching processing takes place for determining, from the time-series correlation, whether the belongings are included in the image data. The matching processing calculates an evaluation value of matching as the value of the evaluation function.

The matching processing can employ, for example, AI. Specifically, a trained inference model is built through machine learning by using a variable template that constantly changes in adapting to a given environmental condition (for example, the luminosity 112), and matching with the belongings is carried out with the time-series correlation as an input. The inference model may be stored inside the vehicle X, for example in the database 109 of the monitoring unit 10, or a mode may be adopted in which the inference model is stored in, for example, a cloud server outside the vehicle X and accessed through wireless communication. Note that a deep learning network such as YOLO (You Only Look Once) can be used as the AI.

For the matching processing, it is preferred to use, for example, lower layer processing that is a processing mode in which a plurality of pieces of data are processed by one instruction from the microprocessor, and higher layer processing that employs programmed control, in combination. In this case, in the lower layer processing, it is preferred to carry out the matching processing by using a local contrast distribution and concentration gradient through the Haar-Like or the HOG characteristics extraction process for low-framerate data, and it is preferred to carry out the matching processing by using a local characteristic amount of edge strength for each edge direction for high-framerate data. Meanwhile, in the higher layer processing, it is preferred to carry out the matching processing by using the AdaBoost procedure that integrates a plurality of characteristic amounts.

In the determination step, whether the belongings are included is determined on the basis of a magnitude relationship between the value of the evaluation function calculated in the synthesis step and the threshold value defined in the threshold value definition step. For example, the belongings are determined to be included in a case in which the value of the evaluation function is greater than the threshold value. Note that, depending on the evaluation function, the belongings may also be determined to be included in a case in which the value of the evaluation function is smaller than the threshold value.

In a case in with the belongings are not determined to be included, it is preferred that the above-described matching processing is carried out with change of the variable template such that the evaluation value of the matching is no less than a predetermined value. In addition, also in a case in which the evaluation value obtained by carrying out the matching processing again after a lapse of a predetermined period of time and adding to the immediately previous matching result is no greater than a predetermined value, it is preferred that the matching processing is carried out with change of the variable template in a similar manner. For example in the above-mentioned detection of a young child, in a case in which the young child is not detected by a template using only the evaluation function of the monitoring camera 102 with a2=0, young child detection performance can be improved by increasing the value of a2 and changing to a template using the evaluation function of the monitoring camera 102 and the evaluation function of the monitoring radar 103 in combination.

The getting-off information 205 can be extracted by using the passenger information 117 and the on-board information 113 of the monitoring information 10a.

Specifically, identification of who is seated in which seat is carried out by way of the passenger information 117. Therefore, when absence of the corresponding passenger in the corresponding seat is determined by the on-board information 113, the passenger can be inferred to have gotten off. Meanwhile, it is more preferred to confirm that the passenger has exited from the vehicle by the outboard information 114. Then, in a case in which it is inferred that all the passengers having boarded have exited, getting-off of all the passengers can be extracted.

### (Detection of Health State)

The vehicle safety assistance system 1 can recognize the health state of a passenger from the on-board information 113, the biological information 115, and the sound information 116. In other words, the recognition information 20a includes health information 206 indicating the health state of each passenger.

Specifically, the vehicle safety assistance system 1 can recognize drowsiness, inattentiveness, and looking away as the health state of the passenger, in addition to a health abnormality. Note that these states may lead to an accident in a case in which the passenger is a driver, and are thus included in the health state in a broad sense.

The health state recognition method of the vehicle safety assistance system 1 may be carried out in a similar way to the extraction method for extracting the belongings detection information 204 in the detection of an object left behind, except for using the three evaluation functions represented by the formulae 3 below.

Drowsiness: Fx=a1×Dx+b1×Vx

Inattentiveness: Fy=a2×Dy+b2×Vy

Looking away: Fz=a3×Dz+b3×Vz 3

In the formulae: Dx, Dy, Dz are evaluation function values obtained from the on-board information 113 and the sound information 116; Vx, Vy, Vz are evaluation function values obtained from the biological information 115; and a1 to a3 and b1 to b3 are weighting variables.

Drowsiness (Dx), inattentiveness (Dy), and looking away (Dz) based on the on-board information 113 and the sound information 116 can be determined from, for example, analysis of the sight line and the behavior of the passenger based on the on-board information 113, or presence/absence and contents of the conversation of the passenger based on the sound information 116, and expressed by 0 (not applicable) and 1 (applicable), or by numerical values therebetween (for example eleven levels by 0.1). Note that AI may be used for this determination.

The biological information 115 includes the breathing rate and the heart rate. Humans are known to be in the states shown in Table 1 depending on the breathing rate and the heart rate. On the basis of these findings, drowsiness (Vx), inattentiveness (Vy), and looking away (Vz) give numerical values in parentheses in Table 1 (from the left, Vx, Vy, Vz).

TABLE 1

| | | Heart Rate (N: Normal Heart Rate) | | |
|---|---|---|---|---|
| | | | | >N + 20 |
| | | | ≥N − 20 | and ≤(220 − |
| | | <N − 20 | and ≤N + 20 | age) × 0.6 |
| Breathing | 10~12 | Vertigo, | Rest, | Normal, |
| Rate | | Gasping, | Drowsiness, | Tension |
| | | Drowsiness | Laxness | (0.2, 0.1, 0.7) |
| | | (0.5, 0.4, 0.1) | (0.5, 0.5, 0) | |
| | 13~22 | Heart disease, | Normal | Activeness, |
| | | Sign of | (0, 0, 0) | Tension, |
| | | drowsiness | | Arousal |
| | | (0.4, 0.4, 0.2) | | (0.1, 0.1, 0.8) |
| | 23~25 | Heart disease, | Activeness, | Activeness, |
| | | Hyperventilation | Tension, | Excitement, |
| | | (0.1, 0.7, 0.2) | Arousal | Stress |
| | | | (0.1, 0.1, 0.8) | (0, 0, 1) |

In Table 1, as N (normal heart rate), a value specific to the passenger may be used if available, and if not available, N=65 may be used. In addition, when the age of the passenger is unknown, 40 years old may be used as a representative value. In regard to the breathing rate as well, Table 1 is compiled on the basis of 18, which is an average; however, when a value specific to the passenger is available, determination may be carried out with the value. The numerical values specific to the passenger may be, for example, recorded in the database 109 in the monitoring unit 10, and referred to.

Note that, in a case in which the biological information 115 does not fall into any of the categories in Table 1, this state is considered to be an abnormal state, and health abnormality is determined regardless of the results of the above formulae 3. In this case, for example, Vx=Vy=Vz=1 can stand.

The weighting variables a1 to a3 and b1 to b3, and the respective threshold values c1, c2, c3 of the evaluation functions Fx, Fy, Fz in the above formulae 3 are defined by the optimization means 202.

Supposing that a1 to a3, b1 to b3, and c1 to c3 are all 0.3 as a standard configuration, in a case in which health abnormality is determined on the basis of information from the biological information 115, it is preferred that a1 to a3=0, and b1 to b3=1, to ensure determination of health abnormality. The same applies to a case in which determination of drowsiness (Dx), inattentiveness (Dy), and looking away (Dz) based on the on-board information 113 and the sound information 116 is not possible. Alternatively, values of a1 to a3, b1 to b3, and c1 to c3 may be adjusted for each passenger.

By thus fusing a plurality of pieces of the monitoring information 10a to provide an evaluation function, recognition accuracy for the state of the passenger can be improved. In addition, a configuration similar to that of the detection of health state enables commonalization of the processing mechanism, leading to reduction in power and cost of the vehicle safety assistance system 1.

### (Identification of Suspicious Person)

The vehicle safety assistance system 1 is capable of recognizing, from the outboard information 114, whether a person approaching the vehicle X from outside is a suspicious person. In other words, the recognition information 20a includes suspicious person identification information 207 identifying, from the outboard information 114, whether the person is a suspicious person.

The recognition of whether a person approaching the vehicle X is a suspicious person may be carried out as follows. First, the ultrasonic sensor 104 detects an object approaching the vehicle X. When a distance between the approaching object and the vehicle X is no greater than a predetermined value, for example 10 m, a suspicious person is identified by the outboard camera 105 and the outboard radar 106. The identification of the suspicious person may be carried out in a manner similar to that of the passenger identification means 110.

### <Control Unit>

The control unit 30 is embodied by, for example, a microcontroller. In addition, the control unit 30 includes an interface unit to the vehicle X, and a communication unit communicating with other outboard instruments such as a mobile phone and a cloud server. Note that a well-known communication means may be used for communication with other instruments. Such a communication means is exemplified by a CAN interface that is capable of communicating without a host computer.

The interface unit is configured to be interfaceable with, for example, a part or all of an on-board display, a horn, lamps, a door locking mechanism, and the like. As a result, for example in a case in which approach of a suspicious person is detected on the basis of the suspicious person identification information 207, control of blaring the horn, blinking the lights, and the like of the vehicle X can be carried out.

The communication unit is configured to be able to communicate with a user's mobile phone or the cloud server via a wireless network. As a result, for example when an object left behind is detected, it is possible to send a message to that effect to the user's mobile phone.

The control unit 30 operates on the basis of the recognition information 20a. Hereinafter, in regard to detection of an object left behind, detection of the health state, and identification of a suspicious person, which are the recognition information 20a recognized by the above-described recognition unit 20, operation of the corresponding control unit 30 is described. Note that the operation of the control unit 30 described below is an example, and other operations may also be employed.

### (Detection of Object Left Behind)

In a case in which it is identified that all the passengers have gotten off the vehicle X on the basis of the getting-off information 205 and that the belongings are present in the vehicle X on the basis of the belongings detection information 204, the belongings can be determined as an object left behind. In this case, the control unit 30 notifies the passenger of the fact that the belongings are present in the vehicle X. The belongings can be inhibited from being left by the control unit 30 notifying the passenger that the belongings are present in the vehicle X.

### (Detection of Health State)

In a case in which drowsiness, inattentiveness, or looking away of a driver among the passengers is confirmed on the basis of the health information 206, the control unit 30 issues an alert to the driver through a message or sound from the on-board display or the horn. This can warn the driver and inhibit occurrence of an accident.

### (Identification of Suspicious Person)

In the identification of a suspicious person, the control unit 30 uses the suspicious person identification information 207. In a case in which the person approaching the vehicle X is identified as a suspicious person on the basis of the suspicious person identification information 207, it is preferred to notify an owner of the vehicle X by the communication unit and intimidate the suspicious person through horn-blaring or lamp-lighting. By thus using the suspicious person identification information 207, prevention of damage to the vehicle or objects in the vehicle is enabled.

To the contrary, in a case in which the person approaching the vehicle X is not a suspicious person, doors may be unlocked or a welcome message may be displayed, and in a case in which the passenger is the driver, a function may be provided for adjusting the position and the height of the seat, the mirror angle, and the like according to suitability to the driver.

In addition, the control unit 30 may include an environment application processing unit that carries out control in adapting to an environmental condition. The environment application processing unit carries out control of the operation modes of the monitoring camera 102 and the monitoring radar 103, waveform formation of digital signals, and the like on the basis of, for example, an environmental condition.

The configuration control of the operation modes changes, for example, exposition on the basis of brightness of a periphery (luminosity 112). In a case in which the vehicle speed or the fall velocity of raindrops during rain can be used, the shutter speed may be increased in proportion thereto. Alternatively, in a case in which temperature can be measured, color temperature of the monitoring camera 102 may be configured on the basis of the peripheral temperature. These are controlled as appropriate on the basis of data usable in the vehicle X. Note that the vehicle speed and the peripheral temperature are information generally available in the vehicle X, and may thus be used. In addition, the fall velocity of raindrops can be acquired by, for example, analysis of an image captured by the outboard camera 105.

Furthermore, the waveform formation is exemplified by edge reinforcement of the image, white balancing, dynamic range expansion, compression and extension, S/N improvement, preprocessing including interface configuration for mutual coordination of instruments, and the like.

### <Control Method of Vehicle Safety Assistance System>

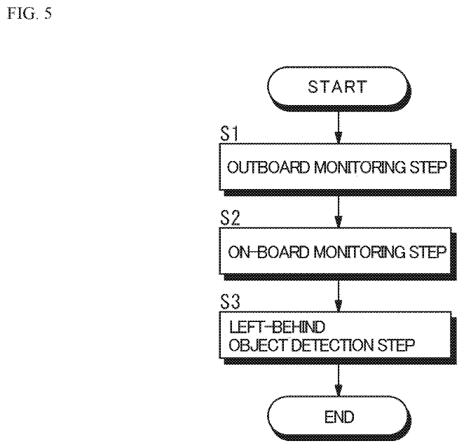

The control method of the vehicle safety assistance system 1 includes, as illustrated in FIG. 5, an outboard monitoring step S1, an on-board monitoring step S2, and a left-behind object detection step S3.

### (Outboard Monitoring Step)

In the outboard monitoring step S1, a suspicious person approaching the vehicle X from the outside of the vehicle is monitored.

The outboard monitoring step S1 is started upon setting to an outboard mode after stopping of the vehicle X and stopping of the engine. The transition to the outboard mode may be set either by the passenger, for example the driver, on board the vehicle X, or automatically upon stopping of the engine as a trigger.

When the outboard monitoring mode is set, the ultrasonic sensor 104 is turned on and detects an object approaching the vehicle X. In a case in which the ultrasonic sensor 104 has detected the approaching object, the recognition unit 20 recognizes whether the person is a suspicious person as described above, and includes the suspicious person identification information 207 in the recognition information 20a.

In a case in which the person approaching the vehicle X is identified as a suspicious person on the basis of the suspicious person identification information 207, the control unit 30, for example, notifies an owner of the vehicle X by the communication unit and intimidates the suspicious person through horn-blaring or lamp-lighting. To the contrary, in a case in which the person approaching the vehicle X is not a suspicious person, the control unit 30 determines, when a distance between the person (passenger) and the vehicle X is a predetermined value, e.g., no greater than 1 m, that the passenger has reached the vehicle X, and may unlock doors or display a welcome message; and in a case in which the passenger is the driver, the control unit 30 adjusts the position and the height of the seat, the mirror angle, and the like according to suitability to the driver.

Furthermore, when all the passengers are determined to be on board on the basis of information from the monitoring camera 102 and the like, the outboard monitoring step S1 is finished and the processing proceeds to an on-board monitoring mode (on-board monitoring step S2).

### (On-Board Monitoring Step)

In the on-board monitoring step S2, a state of the passenger, in particular the driver, is monitored.

In the on-board monitoring step S2, the monitoring camera 102, the monitoring radar 103, the vital sensor 107, and the microphone 108 are turned on to monitor the state of the passenger. Specifically, as described above, behavior of the passenger, in particular the driver, in the vehicle X is monitored using the monitoring camera 102 and the monitoring radar 103, and a change in the health state is detected on the basis of changes in posture and facial complexion of the passenger obtained from the monitoring camera 102 and the monitoring radar 103, information from the vital sensor 107 and the microphone 108, and the like. In this case, for abnormality detection, it is preferred to store individual normal attributes such as the heart rate, the complexion, and the like in, for example, the database 109.

When an abnormality is detected, notification to a predetermined part, distribution of sound and video, and transmission of control information of on-board instruments are carried out by using the communication unit of the control unit 30.

In this step, getting-off of the passenger is also checked, and in a case in which it is confirmed that all the passengers have gotten off, the processing proceeds to the left-behind object detection step S3.

### (Left-Behind Object Detection Step)

In the left-behind object detection step S3, it is detected whether the particular belongings are present in the vehicle X.

The belongings are, for example, a young child, a pet, a bag, a mobile phone, and the like recorded in the database 109. As described above, in a case in which an object is determined to be left behind as a result of detection of the left-behind object by the recognition unit 20, a predetermined terminal is notified via the communication unit of the control unit 30. If no left-behind object is detected, the left-behind object detection step S3 is finished after a lapse of a predetermined time period, for example 10 minutes, and the processing advances to the outboard monitoring step S1. Note that, in a case in which the processing has automatically proceeded to the outboard monitoring step S1 upon stopping of the engine as a trigger and the like, it is possible that the outboard monitoring step S1 is started without waiting for finishing of the left-behind object detection step S3 and both steps are in progress simultaneously. In this case, the left-behind object detection step S3 is finished and the outboard monitoring step S1 is continued.

### ADVANTAGES

The vehicle safety assistance system 1 monitors a passenger in the vehicle X by the monitoring camera 102, recognizes his/her behavior by the recognition unit 20, and carries out control of operation of the vehicle X or notification to the passenger by the control unit 30, whereby safe driving of the vehicle X can be ensured.

### Other Embodiments

The above-described embodiment is not in any way limiting the configuration of the present invention. Therefore, in the above-described embodiment, omission, substitution, or addition of a constitutive element of each part of the above-described embodiment is possible on the basis of description of the present Specification and the common technical knowledge, all of which is construed to be encompassed in the scope of the present invention.

In the above-described embodiment, the case has been described in which the monitoring unit includes the photometer, the monitoring camera, the monitoring radar, the ultrasonic sensor, the outboard camera, the outboard radar, the vital sensor, the microphone, the belongings identification means, the passenger identification means, and the database; however, a part or all of the sensor and the means except for the monitoring camera may be omitted. In the vehicle safety assistance system not employing the monitoring information obtained from the above constitutive elements, appropriate omission is possible.

Alternatively, the monitoring unit may include other monitoring means. Such a monitoring means is exemplified by a radio wave detector that detects radio waves of mobile phones, a GPS device that obtains positional information of mobile phones, and the like. Due to the monitoring unit including the radio wave detector and/or the GPS device, presence of a mobile phone in the vehicle can be easily identified, and in a case in which the belongings likely to be brought into the vehicle include a mobile phone, more reliable detection thereof as an object left behind is enabled. In this case, it is preferred that the optimization means in the recognition unit employs radio field intensity obtained by the radio wave detector and/or GPS information obtained by the GPS device.

In the above-described embodiment, the case has been described in which the monitoring information includes the passenger information identified by the passenger identification means; however, the passenger information is not an essential constitutive element and may be omitted. In other words, the present invention also encompasses a configuration employing only the degree of matching calculated by the passenger identification means.

### INDUSTRIAL APPLICABILITY

As explained in the foregoing, the vehicle safety assistance system according to the present invention enables perception of a state of passengers. Therefore, by employing the vehicle safety assistance system, continuous, safe and certain surveillance of a driver and passengers is enabled from during travel of the vehicle until getting off.

### EXPLANATION OF THE REFERENCE SYMBOLS

1 Vehicle safety assistance system
10 Monitoring unit
10a Monitoring information
20 Recognition unit
20a Recognition information
30 Control unit
101 Photometer
102 Monitoring camera
102a Monitoring region
103 Monitoring radar
103a Monitoring region
104 Ultrasonic sensor
104a Monitoring region
105 Outboard camera
105a Monitoring region
106 Outboard radar
106a Monitoring region
107 Vital sensor
108 Microphone
109 Database
110 Passenger identification means
111 Belongings identification means
112 Luminosity
113 On-board information
114 Outboard information
115 Biological information
116 Sound information
117 Passenger information
118 Degree of matching
119 Belongings information
201 Quantification means
202 Optimization means
203 Recognition information identification means
204 Belongings detection information
205 Getting-off information
206 Health information
207 Suspicious person identification information
X Vehicle

1. A vehicle safety assistance system assisting safety of at least one passenger on board a vehicle, comprising:
a monitoring unit capable of monitoring the at least one passenger in the vehicle;
a recognition unit recognizing a state of the at least one passenger by monitoring information from the monitoring unit; and
a control unit carrying out control of operation of the vehicle or notification to the at least one passenger on basis of recognition information from the recognition unit,
wherein the monitoring unit comprises a monitoring camera.

2. The vehicle safety assistance system according to claim 1, wherein:
the monitoring unit comprises a belongings identification means identifying belongings likely to be brought into the vehicle;
the monitoring information comprises belongings information identified by the belongings identification means;
the recognition information comprises belongings detection information identifying whether the belongings are present in the vehicle and getting-off information identifying whether all of the at least one passenger has gotten off the vehicle; and
when it is identified that all of the at least one passenger has gotten off the vehicle on basis of the getting-off information and that the belongings are present in the vehicle on basis of the belongings detection information, the control unit notifies the at least one passenger that the belongings are present in the vehicle.

3. The vehicle safety assistance system according to claim 2, wherein the monitoring unit comprises a monitoring radar.

4. The vehicle safety assistance system according to claim 3, wherein the belongings identification means uses periodic oscillation detected by the monitoring radar.

5. The vehicle safety assistance system according to claim 1, wherein the recognition unit comprises:
a quantification means expressing the state of the at least one passenger by a numerical value from the monitoring information through an evaluation function;
an optimization means optimizing a threshold value for identifying the state of the at least one passenger according to a magnitude relationship with the numerical value; and
a recognition information identification means identifying the state of the at least one passenger as the recognition information from the numerical value and the threshold value.

6. The vehicle safety assistance system according to claim 5, wherein:
the monitoring unit comprises: a database on which a reference image of a passenger captured beforehand is recorded; and a passenger identification means identifying the passenger by comparatively searching for a captured image of the passenger captured by the monitoring camera with the reference image;
the passenger identification means is configured to be capable of calculating a degree of matching between the captured image and the reference image; and
the optimization means of the recognition unit uses the degree of matching.

7. The vehicle safety assistance system according to claim 5, wherein:
the monitoring unit comprises a photometer measuring luminosity of a periphery of the vehicle; and
the optimization means of the recognition unit uses the luminosity.

8. The vehicle safety assistance system according to claim 1, wherein:
the monitoring unit comprises a vital sensor acquiring biological information of the at least one passenger; and
the monitoring information comprises the biological information obtained from the vital sensor.

9. The vehicle safety assistance system according to claim 1, wherein:
the monitoring unit comprises an ultrasonic sensor capable of detecting a person approaching the vehicle from outside of the vehicle, and an outboard camera or an outboard radar for sensing the person;
the monitoring information comprises outboard information obtained from the ultrasonic sensor, and the outboard camera or the outboard radar;
the recognition information comprises suspicious person identification information identifying, from the outboard information, whether the person is a suspicious person; and
the control unit uses the suspicious person identification information.

10. The vehicle safety assistance system according to claim 2, wherein the recognition unit comprises:
a quantification means expressing the state of the at least one passenger by a numerical value from the monitoring information through an evaluation function;
an optimization means optimizing a threshold value for identifying the state of the at least one passenger according to a magnitude relationship with the numerical value; and
a recognition information identification means identifying the state of the at least one passenger as the recognition information from the numerical value and the threshold value.

11. The vehicle safety assistance system according to claim 3, wherein the recognition unit comprises:
a quantification means expressing the state of the at least one passenger by a numerical value from the monitoring information through an evaluation function;
an optimization means optimizing a threshold value for identifying the state of the at least one passenger according to a magnitude relationship with the numerical value; and
a recognition information identification means identifying the state of the at least one passenger as the recognition information from the numerical value and the threshold value.

12. The vehicle safety assistance system according to claim 4, wherein the recognition unit comprises:
a quantification means expressing the state of the at least one passenger by a numerical value from the monitoring information through an evaluation function;
an optimization means optimizing a threshold value for identifying the state of the at least one passenger according to a magnitude relationship with the numerical value; and
a recognition information identification means identifying the state of the at least one passenger as the recognition information from the numerical value and the threshold value.

13. The vehicle safety assistance system according to claim 10, wherein:
the monitoring unit comprises: a database on which a reference image of a passenger captured beforehand is recorded; and a passenger identification means identifying the passenger by comparatively searching for a captured image of the passenger captured by the monitoring camera with the reference image;
the passenger identification means is configured to be capable of calculating a degree of matching between the captured image and the reference image; and
the optimization means of the recognition unit uses the degree of matching.

14. The vehicle safety assistance system according to claim 11, wherein:
the monitoring unit comprises: a database on which a reference image of a passenger captured beforehand is recorded; and a passenger identification means identifying the passenger by comparatively searching for a captured image of the passenger captured by the monitoring camera with the reference image;
the passenger identification means is configured to be capable of calculating a degree of matching between the captured image and the reference image; and
the optimization means of the recognition unit uses the degree of matching.

15. The vehicle safety assistance system according to claim 12, wherein:
the monitoring unit comprises: a database on which a reference image of a passenger captured beforehand is recorded; and a passenger identification means identifying the passenger by comparatively searching for a captured image of the passenger captured by the monitoring camera with the reference image;
the passenger identification means is configured to be capable of calculating a degree of matching between the captured image and the reference image; and
the optimization means of the recognition unit uses the degree of matching.

16. The vehicle safety assistance system according to claim 10, wherein:
the monitoring unit comprises a photometer measuring luminosity of a periphery of the vehicle; and
the optimization means of the recognition unit uses the luminosity.

17. The vehicle safety assistance system according to claim 11, wherein:
the monitoring unit comprises a photometer measuring luminosity of a periphery of the vehicle; and
the optimization means of the recognition unit uses the luminosity.

18. The vehicle safety assistance system according to claim 12, wherein:
the monitoring unit comprises a photometer measuring luminosity of a periphery of the vehicle; and
the optimization means of the recognition unit uses the luminosity.
